(19) United States
(12) Patent Application Publication
(10) Pub. No.: US 2015/0231291 A1
(43) Pub. Date: Aug. 20, 2015

(54) Methods and Systems for a Secondary Sanitization Fluid Path

(71) Applicant: StethoSwipe LLC, Boston, MA (US)

(72) Inventors: Justin ZAGHI, Tarzana, CA (US); Scott Albert LITTLEFIELD, Stow, MA (US); Kiran AGARWAL-HARDING, Clarksville, MD (US)

(73) Assignee: STETHOSWIPE LLC, Boston, MA (US)

(21) Appl. No.: 14/625,451

(22) Filed: Feb. 18, 2015

Related U.S. Application Data
(60) Provisional application No. 61/941,224, filed on Feb. 18, 2014.

(51) Int. Cl. A61L 2/18

(52) U.S. Cl. CPC A61L 2/18

(57) ABSTRACT

Techniques for providing a secondary path for sanitization fluid are disclosed. In one particular embodiment, the techniques may be realized as an apparatus providing an additional sanitization fluid dispensing path. The additional sanitization fluid dispensing path may include a sealing housing which may include a sealing ring, the sealing ring having a first opening for holding a first sanitization fluid dispensing path, a perimeter ring wall connected at one end to the sealing ring and arranged substantially perpendicular to the sealing ring, wherein the perimeter ring wall is configured to fasten the sealing ring to a sanitization fluid container, and a sealed cavity configured to accept an adapter to conduct sanitization fluid, wherein acceptance of the adapter opens the sealed cavity to form a second sanitization fluid dispensing path.

### CROSS-REFERENCE TO RELATED APPLICATIONS

This patent application claims priority to U.S. Provisional Patent Application No. 61/941,224, filed Feb. 18, 2014, which is hereby incorporated by reference herein in its entirety.

### TECHNICAL FIELD

The present disclosure relates generally to techniques for sanitizing medical instruments and, more particularly, to techniques for improving sanitization of stethoscopes.

### BACKGROUND

Several studies have shown that stethoscope membranes harbor disease-causing bacteria, including methicillin-resistant Staphylococcus aureus (MRSA) and vancomycin-resistant enterococci (VRE). In a study of 300 stethoscopes, 87% were contaminated with pathogenic bacteria. VRE contamination rates have been as high as 31%, and MRSA has been found on up to 15% of healthcare workers' stethoscopes. S. aureus colonies can survive on stethoscope membranes for longer than 18 hours. Without proper disinfection, stethoscopes represent a potential vector of transmission of these organisms from one patient to another.

Routine disinfection of stethoscopes may reduce the potential for transmission by reducing the burden of contamination. Bacterial counts on stethoscope diaphragms can be reduced by 95% using alcohol-based disinfectant products, by 90% using non-ionic detergents, and by 75% using antiseptic soaps. More recently, alcohol-based hand rubs were shown to reduce bacterial contamination on stethoscopes by approximately 90%. Despite the availability of effective disinfectants, only a minority of healthcare providers regularly disinfects their stethoscope. In a survey of 150 healthcare workers in 1995, 48% of respondents reported cleaning their stethoscope daily or weekly. A 1999 study by Bernard et al. found that only 22% of users regularly disinfect their stethoscope.

### SUMMARY

The present disclosure will now be described in more detail with reference to particular embodiments thereof as shown in the accompanying drawings. While the present disclosure is described below with reference to particular embodiments, it should be understood that the present disclosure is not limited thereto. Those of ordinary skill in the art having access to the teachings herein will recognize additional implementations, modifications, and embodiments, as well as other fields of use, which are within the scope of the present disclosure as described herein, and with respect to which the present disclosure may be of significant utility.

Techniques for stethoscope sanitation are disclosed. In one particular embodiment, the techniques may be realized as a system for stethoscope sanitation including a first dispensing nozzle positioned to dispense a sanitization fluid on a first surface of a stethoscope, a second dispensing nozzle positioned to dispense a sanitization fluid on a second surface of a stethoscope, a first sensor configured to detect the presence of the stethoscope within a close proximity of the first dispensing nozzle and to detect the presence of the stethoscope within a close proximity of the second dispensing nozzle, and a pump configured to receive sanitization fluid from a sanitization fluid receptacle and to provide the sanitization fluid to the first dispensing nozzle and the second dispensing nozzle. The techniques may include a microcontroller configured to receive a signal from the first sensor indicating the presence of the stethoscope within close proximity of the first dispensing nozzle and the second dispensing nozzle, and control pumping of the sanitization fluid by the pump, wherein pumping is initiated in response to reception of the signal.

In accordance with other aspects of this particular embodiment, the microcontroller may be configured to control pumping of the sanitization fluid to pump a specified amount of sanitization fluid.

In accordance with further aspects of this particular embodiment, the microcontroller may be configured to control pumping of the sanitization fluid to pump for a specified period of time.

In accordance with additional aspects of this particular embodiment, the microcontroller may be configured to control pumping of the sanitization fluid to pump sanitization fluid for a specified number of pump cycles.

In accordance with additional aspects of this particular embodiment, the specified period of time may be configurable.

In accordance with additional aspects of this particular embodiment, the techniques may include a second sensor configured to detect the presence of the stethoscope within a close proximity of the first dispensing nozzle and to detect the presence of the stethoscope within a close proximity of the second dispensing nozzle.

In accordance with additional aspects of this particular embodiment, the first dispensing nozzle and the second dispensing nozzle may be positioned within an elliptically shaped channel having an opening on a horizontal side wall of the channel permitting insertion of a portion of the stethoscope for sanitization.

In accordance with additional aspects of this particular embodiment, the opening may be positioned to improve placement of a stethoscope portion between the first dispensing nozzle and the second dispensing nozzle.

In accordance with additional aspects of this particular embodiment, at least one of the first dispensing nozzle and the second dispensing nozzle may be recessed within the channel to reduce a likelihood of contact with the stethoscope.

In accordance with additional aspects of this particular embodiment, the first dispensing nozzle may be located on an upper portion of the elliptical channel, the second dispensing nozzle may be located on a lower portion of the elliptical channel, and the opening may be positioned between the first dispensing nozzle and the second dispensing nozzle permitting simultaneous sanitization of a first side of a stethoscope portion and a second side of stethoscope.

In accordance with additional aspects of this particular embodiment, the first sensor may be located on an upper portion of the elliptical channel.

In accordance with additional aspects of this particular embodiment, the system may include a supply tube for integrating the stethoscope sanitization system with a hand sanitization system, wherein the supply tube may be configured to receive sanitization fluid from a sanitization fluid receptacle of the hand sanitization system.

In accordance with additional aspects of this particular embodiment, the microcontroller may be configured to log one or more events.

In accordance with additional aspects of this particular embodiment, event log data may include one or more of the following: an event name, an event id, an event date, an event time, and a system id.

In accordance with additional aspects of this particular embodiment, logged events may include at least one of: a number of activations of one or more stethoscope sanitization components, a number of activations of one or more hand sanitization components, an amount of sanitization fluid used by the stethoscope sanitization system, an amount of sanitization fluid used by the hand sanitization system, a fluid level, a battery level, a number of pump iterations, and a power level.

In accordance with additional aspects of this particular embodiment, the system may include one or more indicators.

In accordance with additional aspects of this particular embodiment, the microcontroller may be configured to calculate a ratio of activations of one or more hand sanitization components to activations of one or more stethoscope sanitization components.

In accordance with additional aspects of this particular embodiment, the microcontroller may be further configured to send an alert utilizing the one or more indicators.

In accordance with additional aspects of this particular embodiment, the alerts may include at least one of: a visual alert reminding a user to sterilize a stethoscope based on a ratio of activations of one or more hand sanitization components to activations of one or more stethoscope sanitization components and an audible alert based on a ratio of activations of one or more hand sanitization components to activations of one or more stethoscope sanitization components.

In accordance with additional aspects of this particular embodiment, the alert may include at least one of: an indicator of a power level, an indicator of a fluid level, and a fault indicator.

In accordance with additional aspects of this particular embodiment, the first sensor may include at least one of: an active infrared sensor, a passive infrared sensor, a photoelectric sensor, and an ultrasonic sensor.

In another particular embodiment, the techniques may be realized as a method of stethoscope sanitation. The method may include detecting, using a first sensor a presence of the stethoscope within a close proximity of a first dispensing nozzle and a second dispensing nozzle, detecting, using a second sensor a presence of the stethoscope within a close proximity of a first dispensing nozzle and a second dispensing nozzle, determining by a processor that a portion of a stethoscope is positioned within a specified range of at least one of the first dispensing nozzle and the second dispensing nozzle, wherein the determination is based at least in part on the input received from the first sensor and the second sensor; and initiating dispensing of sanitization liquid from the first dispensing nozzle and the second dispensing nozzle based on the determination by the processor, wherein the dispensing of the sanitation liquid is configurable.

### BRIEF DESCRIPTION OF FIGURES

In order to facilitate a fuller understanding of the present disclosure, reference is now made to the accompanying drawings, in which like elements are referenced with like numerals. These drawings should not be construed as limiting the present disclosure, but are intended to be illustrative only.

FIG. 1 shows a front view of a system for sanitization of stethoscopes, in accordance with an embodiment of the present disclosure.

FIG. 2 shows a back view of a system for sanitization of stethoscopes, in accordance with an embodiment of the present disclosure.

FIG. 3 depicts sensor detection of a stethoscope portion in a system for sanitization of stethoscopes, in accordance with an embodiment of the present disclosure.

FIG. 4 illustrates sanitizing fluid dispersion in a system for sanitization of stethoscopes, in accordance with an embodiment of the present disclosure.

FIG. 5 shows a pump for sanitization of stethoscopes, in accordance with an embodiment of the present disclosure.

FIG. 6 shows a microcontroller for sanitization of stethoscopes, in accordance with an embodiment of the present disclosure.

FIG. 7 depicts a fitting for attaching a system for sanitization of stethoscopes to a sanitizing fluid receptacle, in accordance with an embodiment of the present disclosure.

FIG. 8 depicts a fitting for attaching a system for sanitization of stethoscopes to a sanitizing fluid receptacle, in accordance with an embodiment of the present disclosure.

FIG. 9 shows a block diagram for a system sanitization of stethoscopes, in accordance with an embodiment of the present disclosure.

FIG. 10 is a flowchart depicting a method for sanitization of stethoscopes in accordance with an embodiment of the present disclosure.

FIG. 11 is a cutaway view of an apparatus providing an additional sanitization fluid dispensing path in accordance with an embodiment of the present disclosure.

FIG. 12 is a cutaway view of an apparatus providing an additional sanitization fluid dispensing path in accordance with an embodiment of the present disclosure.

FIG. 13 is an illustration of an apparatus providing an additional sanitization fluid dispensing path in accordance with an embodiment of the present disclosure.

FIG. 14 is a schematic of a sanitization fluid adapter in accordance with an embodiment of the disclosure.

FIG. 15 is an exploded view of an apparatus providing an additional sanitization fluid dispensing path in accordance with an embodiment of the present disclosure.

FIG. 16 is an illustration of a sanitization apparatus including an apparatus providing an additional sanitization fluid dispensing path in accordance with an embodiment of the present disclosure.

### DETAILED DESCRIPTION

According to some embodiments, a stethoscope sanitization system may consist of a housing (e.g., plastic, metal, etc.), containing a detection system (e.g., infrared) that signals a microcontroller pumping system, for the dispensing of a sterile fluid onto two opposing surfaces of a stethoscope simultaneously. In some embodiments, the system may complement an existing hand sanitizing system, and utilize the hand sanitizer's fluid cartridges. In some embodiments, the system may be independent of a hand sanitizing system.

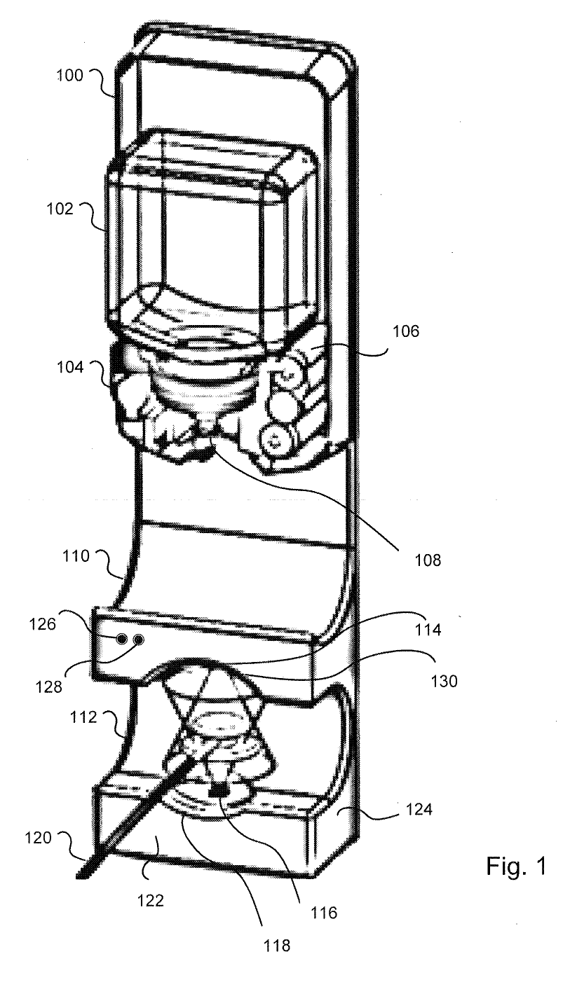

FIG. 1 shows a front view of a system 100 for sanitization of stethoscopes, in accordance with an embodiment of the present disclosure. FIG. 1 depicts an exemplary device layout. A hand sanitizing system may be mated to the device back plate via standard mounting hole configuration. The front of the device may contain a drip tray 110 for the hand sanitizer, and the stethoscope dome 130. The stethoscope dome 130 may contain one or more the infrared sensors for detection of the stethoscope and misting nozzles 114 and 116 for cleaning of the stethoscope. The shape of stethoscope dome 130 may acts as a visual cue, guiding the end user where to place the stethoscope 120 in elliptical channel 112 for cleaning. The misting nozzles 114 and 116 may be recessed in domes 130 and 118. This may reduce a likelihood of contact between a stethoscope 120 and the system 100 and may improve a placement of stethoscope 120 between misting nozzles 114 and 116.

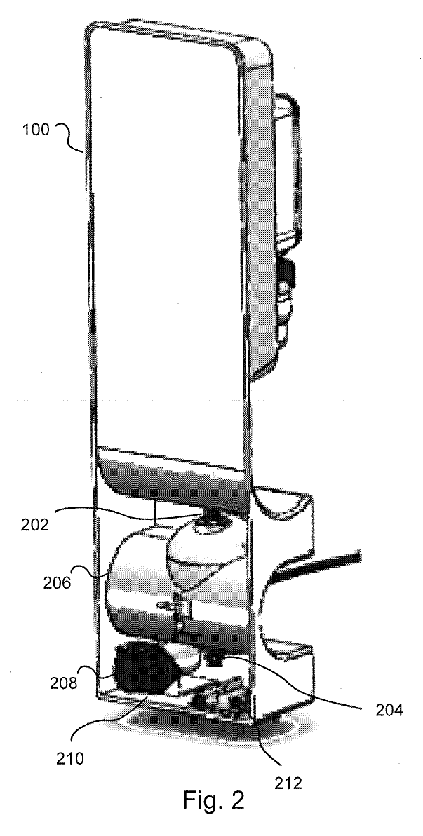

System 100 may include one or more indicators 126 and 128. Indicators 126 and 128 may be LEDs or other lights for providing visual indicators. System 100 may contain one or more elements for providing other alerts (e.g., buzzers, bells, an LCD screen, etc.). Alerts or alarms may be controlled by microcontroller 212 of FIG. 2 and may include one or more of a visual alert reminding a user to sterilize a stethoscope based on a ratio of activations of one or more hand sanitization components to activations of one or more stethoscope sanitization components, an audible alert based on a ratio of activations of one or more hand sanitization components to activations of one or more stethoscope sanitization components, an indicator of a power level, an indicator of a fluid level, and a fault indicator. For example, microcontroller 212 may log detected events such as for example, a number of activations of one or more stethoscope sanitization components, a number of activations of one or more hand sanitization components, an amount of sanitization fluid used by the stethoscope sanitization system, an amount of sanitization fluid used by the hand sanitization system, a fluid level, a battery level, a number of pump iterations, and a power level. Events log data may include one or more of an event name, an event id, an event date, an event time, and a system ID. Based on the detection of multiple iterations of hand sanitization without an activation of stethoscope sanitization an alert may be triggered. For example, if hand sanitization fluid is dispensed three times without a single stethoscope sanitization, an alert may be triggered to remind users to sanitize their stethoscope. In some embodiments, once a single sanitization is triggered (i.e., either hand sanitization or stethoscope sanitization) the other sanitization system may blink an LED or display some other indicator until both have been completed. For example, if a user sanitizes their hands an LED above a hand sanitization component may display green, but a LED above a stethoscope sanitization components may blink or display red or yellow. Once a user activates the stethoscope sanitization components, an LED above the stethoscope sanitization components may turn off, a green LED may display, an LED may stop blinking and display in a solid state, or another acknowledgement may be displayed or sounded.

Access to one or more internal elements of system 100 such as, for example, a battery compartment, may be provided through an access door, slide out drawer, or other entry (e.g., via side panel 124 or front panel 122).

As shown in FIG. 1, system 100 may be integrated with a hand sanitization system (shown as a sectional view to illustrate placement of motor 104, batteries 106, and dispensing nozzle 108). In some embodiments, integration of a system 100 with a hand sanitization system may enable sharing of one or more components such as, for example, a fluid reservoir, a battery, a microcontroller, a pump, a sensor, or other components. As illustrated in FIG. 1 and described in further detail with reference to FIGS. 2, 7, and 8 below, system 100 may share a common sanitization fluid reservoir 102 which may be accessed by fluid plumbing. In some embodiments, system 100 may contain only stethoscope sanitization components and may not be integrated with a hand sanitization station.

FIG. 2 shows a back view of a system 100 for sanitization of stethoscopes, in accordance with an embodiment of the present disclosure. The back side of the device provides space for the mounting of the pump assembly 208, the microcontroller PCB 212 and fluid plumbing (not shown) that tap the fluid supply. Sensors 202 and 204 on stetho-dome 206 may be operatively connected to microcontroller 212 and may receive power from a battery in battery compartment 210. Sensors 202 and 204 may detect the presence of a stethoscope within stethoscope dome 130 and may provide signals and data to microcontroller 212. Pump 208 may be operatively connected to microcontroller 212 and may receive power from a battery in battery compartment 210. Pump 208 may receive sanitization fluid from sanitization fluid reservoir 102 and may supply sanitization fluid to misting nozzles 114 and 116 under the control of microcontroller 212.

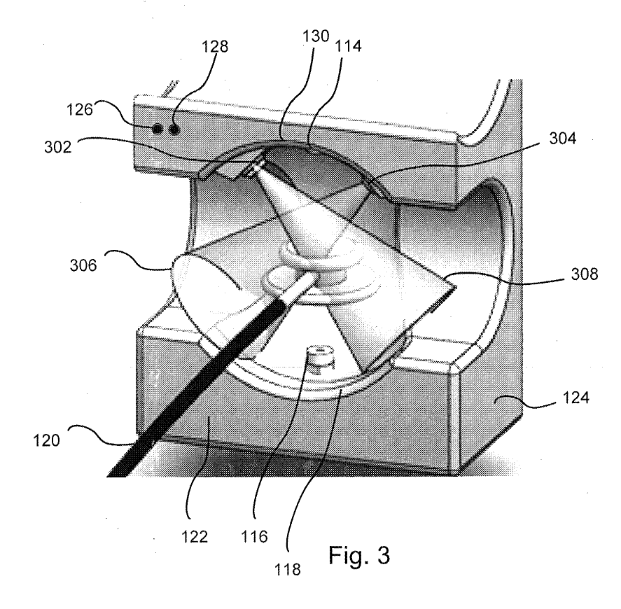

FIG. 3 depicts sensor detection of a stethoscope portion in a system for sanitization of stethoscopes, in accordance with an embodiment of the present disclosure. When the stethoscope is introduced into the path of one or more sensors (e.g., active infrared detector and emitter, passive infrared detector, a photoelectric sensor, and/or an ultrasonic sensor), there is a change in signal reflection, sending a state change to the microcontroller 212. The pump system 208 may then be activated, which pushes fluid to the misting nozzles 114 and 116, rinsing the surface of the stethoscope 120. The positioning of misting nozzles 114 and 116 may allow for simultaneous sanitization of both sides of stethoscope. As indicated in FIG. 3, the fields of detection 308 and 306 of sensors 302 and 304, respectively, may provide a co-incident region which may be located in an optimized location for stethoscope sanitization. According to some embodiments, a first indicator may indicate to a user that a stethoscope has been detected and a second indicator may indicate to a user that a stethoscope is placed correctly. For example if a user places a stethoscope within a stetho-dome but off-center indicator 126 may display yellow. If a stethoscope is placed properly between misting nozzles 114 and 116 indicator 128 may display green. Misting nozzle 116 may protrude above a surface of dome 118 to avoid a buildup of sanitization liquid. Sensors 302 and 304 may be placed on dome 130 a sufficient distance from misting nozzle 114 to avoid significant collection of sanitization fluid. Placement of sensors 302 and 304 may be designed to improve a location co-incident region of detection such that placement of a stethoscope may be optimized for sanitization.

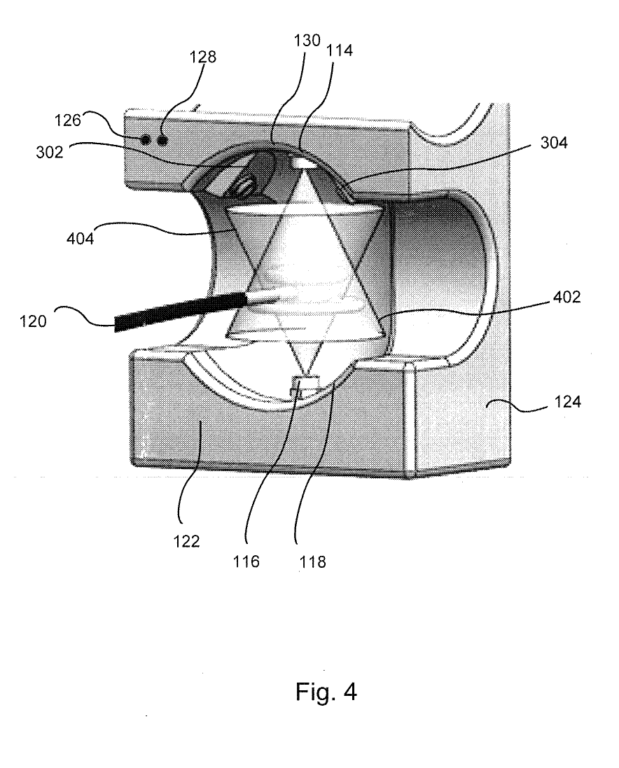

FIG. 4 illustrates sanitizing fluid dispersion in a system for sanitization of stethoscopes, in accordance with an embodiment of the present disclosure. Misting nozzles 114 and 116 may provide sanitization fluid dispersal patterns 402 and 404, which may be designed to sufficiently and simultaneously coat a stethoscope for sanitization.

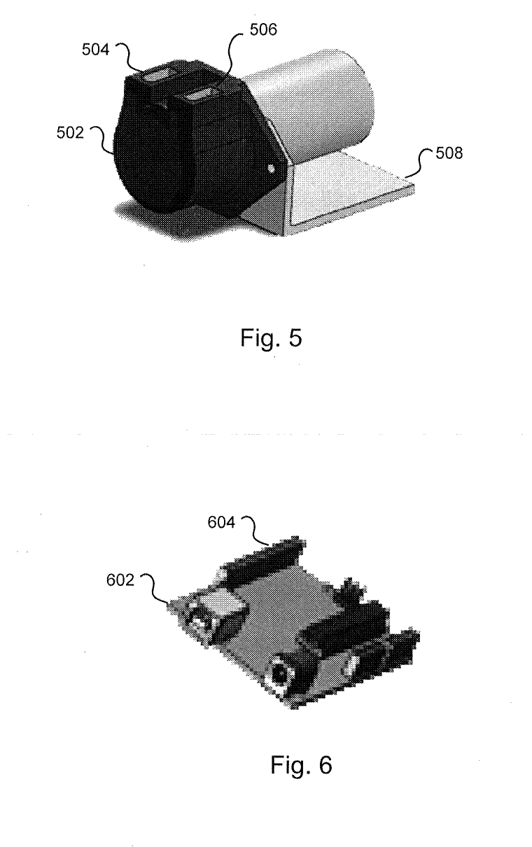

FIG. 5 shows a pump 502 for sanitization of stethoscopes, in accordance with an embodiment of the present disclosure. Pump 502 may contain a fluid input port 504 and an output port 506 and mounting bracket 508. In some embodiments, pump 502 may be self-priming. According to some embodiments pump 502 may be either peristaltic or a small gear pump, with a low operating pressure. A pump motor may be able to be operated on DC voltage to allow a device to be battery operated. Fluid connectors may be standard push on barb fittings medical grade. Fluid run to the pump may be assisted by gravity flow, and the exit path may divide to two or more spraying nozzles (e.g., misting nozzles 114 and 116). Power for the motor side of the relay control may be provided by a battery pack (e.g., an off the shelf battery with a range between 6 and 9 volts). Pump 502 may be controlled by microcontroller 212 to control pumping of the sanitization fluid to pump a specified amount of sanitization fluid, to control pumping of the sanitization fluid to pump for a specified period of time, and/or to control pumping of the sanitization fluid to pump sanitization fluid for a specified number of pump cycles. Because the pump may be microcontroller controlled, the pump on and off time can be controlled for optimal surface wetting of the stethoscope. The microcontroller can be tuned to register distance in regard to the sensor array and based on an averaging of the distance tune the pump cycling. Pump duration time can be preset for standard operation.

FIG. 6 shows a microcontroller 602 for sanitization of stethoscopes, in accordance with an embodiment of the present disclosure. As discussed above, microcontroller 602 and/or 212 may be operatively connected to a pump, one or more sensors, one or more indicators, a battery, and/or other components (e.g., components of a hand sanitization system or a microcontroller of a hand sanitization system for collection of data). Microcontroller 602 may contain electronic storage (e.g., read only memory or a random access memory or both). Microcontroller 602 may include by way of example semiconductor memory devices, (e.g., EPROM, EEPROM, and flash memory devices) or other storage. Microcontroller 602 may contain a processor and/or special purpose logic circuitry. Microcontroller 602 may utilize electronic storage for instructions (e.g., software), data logging, and other purposes. Microcontroller 602 may be mounted on a PCB 604 which may contain one or more connections and/or interfaces for exchange of data, power, and other purposes. Other components may be placed on PCB 604.

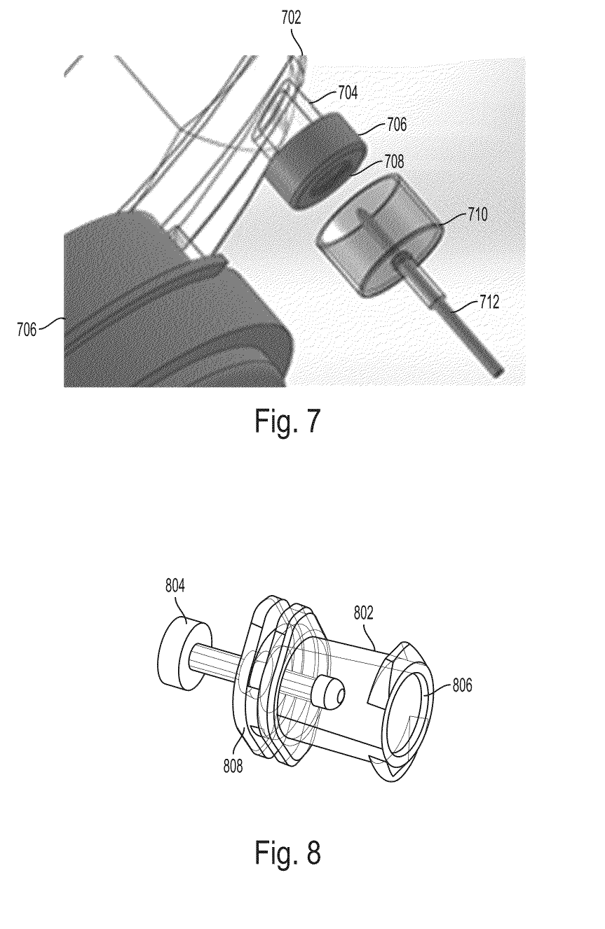

FIG. 7 depicts a fitting for attaching a system for sanitization of stethoscopes to a sanitizing fluid receptacle, in accordance with an embodiment of the present disclosure. An interface with fluid receptacle 702 can be through multiple methods. For example, a fluid receptacle manufacturer can provide a port 704 on their bottle either thru a mold extrusion thermal sealing or UV bonding. The interface may consists of a rubber silicon “Septa” style seal 708 and a crimp on metallic cap 706 used in standard, laboratory practice. The piercing of the septa membrane may be through a shrouded needle housing 710 that is a slip fit over the Septa cap and seal. The shroud provides safety housing for a piercing needle 712. This method will work with standard luer style fittings as well. Medical grade silicon tubing may be UV bonded to the needle shroud housing, for fluid migration.

FIG. 8 depicts a fitting for attaching a system for sanitization of stethoscopes to a sanitizing fluid receptacle, in accordance with an embodiment of the present disclosure. FIG. 8 depicts a variation, consisting of a standard luer style fitting 802. Internal to the female Luer fitting would be a spring loaded plunger 804. The spring shall be configured that in the normal rest configuration the plunger with a seal would set against the back side of the luer fitting 808, preventing fluid leakage. Upon installation of the mating tubing connector 806 the plunger would be forced upward allowing fluid to migrate to the pump mechanism. Medical grade silicon tubing may be UV bonded to the mating adapter.

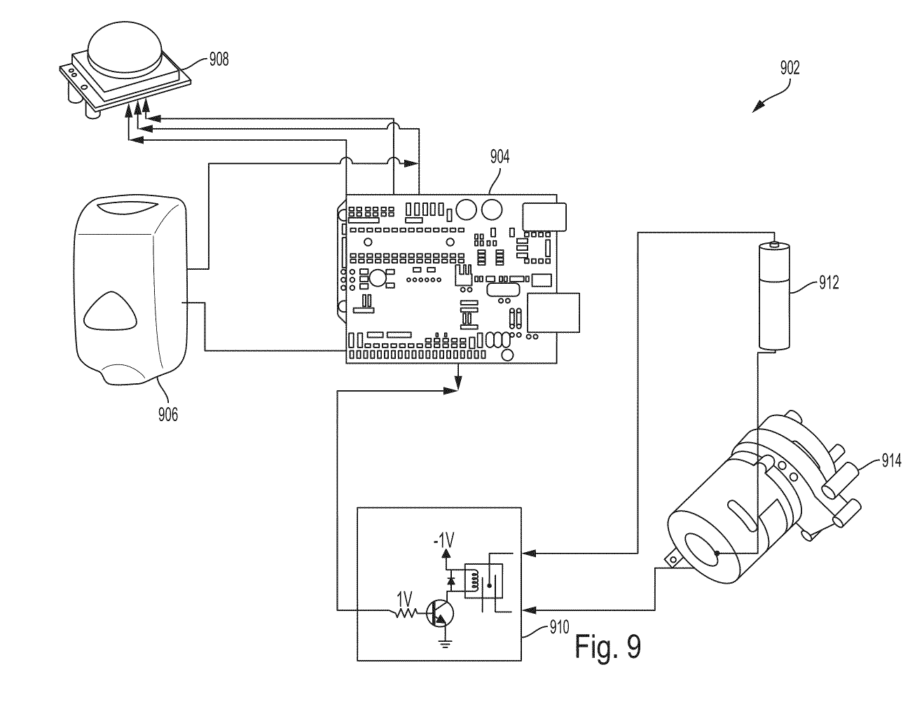

FIG. 9 shows a block diagram for a system sanitization of stethoscopes, in accordance with an embodiment of the present disclosure. An opto-coupled relay circuit 910 may isolate the detection circuitry from the pump and fluid controls. A microcontroller on circuit board 904 allows for the device to be programmed for pump on and off time, as well as the level and sensitivity of the detection circuitry. The circuit board 904 may contain logic and storage configured to data log stethoscope and hand swipes for data collection. Firmware may control timing, pump cycle time, detector sensitivity, and alert notifications, and other operation aspects (e.g., pump priming on power up). Firmware may reside in the static memory of the microcontroller and may boot upon power up.

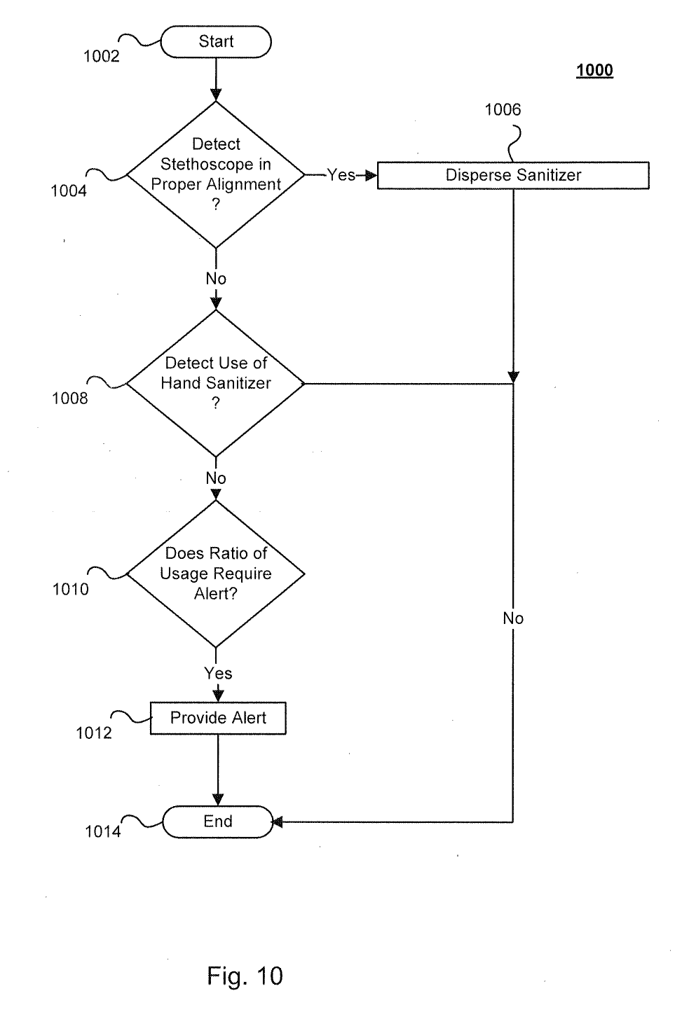

FIG. 10 is a flowchart depicting a method 1000 for sanitization of stethoscopes in accordance with an embodiment of the present disclosure. At block 1002, the method 1000 may begin.

At block 1004, it may be determined whether a stethoscope or other medical instrument has been detected. According to some embodiments, alignment of a stethoscope or other instrument within a specified region may be required. If a stethoscope or other instrument is detected the method may proceed to block 1006.

At block 1006 sanitizing fluid may be dispensed. Fluid may be dispensed from one or more nozzles. In some embodiments, two nozzles may be utilized in a position allowing coating of two sides of a stethoscope or other medical instrument simultaneously. An amount of fluid dispensed may be configurable and may be based upon input received may a processor from one or more sensors (e.g., a position of an instrument and/or the presence or absence of an instrument within a detectable region).

At block 1008 it may be determined whether one or more hand sanitizing components have been utilized. If one or more hand sanitizing components have been utilized, the method may proceed to block 1010. If hand sanitization has not been detected, the method may end at block 1014.

At block 1010 the method may determine whether a ratio of hand sanitizing to stethoscope sanitizing exceeds a specified configurable threshold (e.g., three hand sanitizing detections without a single stethoscope sanitization). If the ratio of usage requires an alert the method may proceed to block 1012. If the ratio of usage does not require an alert the method may end at block 1014.

At block 1012, an alert may be provided. An alert may be audible (e.g., via a buzzer, speaker, bell, etc.) and/or visible (e.g., via an LED or an LCD display). An alert may remind a user to sterilize a stethoscope or other medical instruments.

At block 1014, the method 1000 may end.

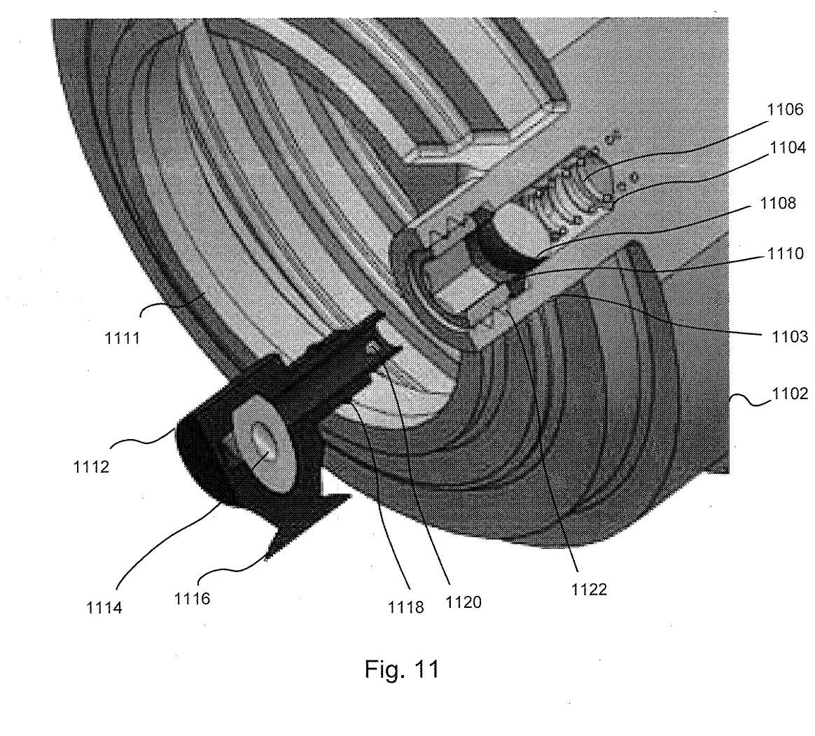

FIG. 11 is a cutaway view of an apparatus providing an additional sanitization fluid dispensing path in accordance with an embodiment of the present disclosure. As illustrated in FIG. 11, sealing housing 1102 may contain an opening 1111 which may accommodate a primary path for sanitization fluid. However, sealing housing 1102 may also contain one or more secondary paths for dispensing sanitization fluid such as secondary fluid path 1103. Secondary fluid path 1103 may allow sealing housing 1102 to adapt a fluid receptacle (e.g., fluid receptacle 102 of FIG. 1) so that a single fluid receptacle may provide fluid for multiple dispensing areas of a sanitization apparatus. For example, a primary path for sanitization fluid may provide sanitization fluid for hand sanitization and secondary fluid path 1103 may provide access to sanitization fluid for a dispensing area designed to sanitize medical instruments (e.g., stethoscopes). According to some embodiments, sealing housing 1102 may be a modified plastic housing.

Secondary fluid path 1103 may be a sanitization fluid conduit that may be initially sealed. In some embodiments, secondary fluid path 1103 may be sealed using a puncturable covering or sealing membrane. For example, a fluid receptacle manufacturer may use a rubber silicon “Septa” style seal and/or a crimp on metallic cap. The piercing of the septa membrane may be through a shrouded needle housing that is a slip fit over the cap and seal. The shroud may provide safety housing for a piercing needle.

In some embodiments, secondary fluid path 1103 may be sealed via a sealing stopper 1108 which may be configured to recede back into conduit 1104 upon acceptance of tubing adapter 1112. For example, sealing stopper 1108 may be a ball bearing (e.g., stainless steel or polyethylene) which may be held against a sealing ring 1110 (e.g., a gasket or O-ring). In some embodiments, sealing stopper 1108 may be held in place against sealing ring 1110 by spring 1106 or another mechanical device. Insertion of tubing adapter 1112 into conduit 1104 may push sealing stopper 1108 back into conduit 1104 providing conduit 1114 of tubing adapter 1112 access to sanitization fluid via one or more apertures 1120. Tubing adapter 1112 may secure into place via locking ring 1118 engaging one or more grooves 1122 of conduit 1104. Sanitization fluid may flow from a receptacle to which sealing housing 1102 is secured via conduit 1104 and one or more apertures 1120 to conduit 1114 of tubing adapter 1112 and out of the tubing adapter 1112 to mating adapter 1116. Mating adapter 1116 may be bonded or otherwise connected to tubing or other sanitization fluid conduits (e.g., UV bonded to medical grade silicon tubing). Conduit attached to mating adapter 1116 may conduct sanitization fluid to one or more separate dispensers (e.g., provide additional sanitization functionality beyond hand sanitization such as sanitization of medical instruments).

In some embodiments, secondary fluid path 1103 may be sealed and opening of the secondary fluid path may be via an adapter that screws into place and punctures a membrane or causes a stopper to recede. In one or more embodiments, secondary fluid path 1103 may be sealed by a stopper designed to be broken or detached by insertion of a tubing adapter. For example, a stopper may be attached to an inner wall of a channel or conduit 1104 to form a seal and the sealing stopper may be configured to break an attachment to the inner wall and to recede into the conduit or channel upon the acceptance of the adapter into the conduit. For example, a stopper may be a plastic stopper which may be thinner along a perimeter boundary attaching the stopper to the inner wall and may be designed to separate and recede upon insertion of a tubing adapter.

In some embodiments, secondary fluid path 1103 may be resealable (e.g., removal of tubing adapter 1112 may allow a stopper to seal against a sealing ring). In some embodiments, secondary fluid path 1103 may not be resealable.

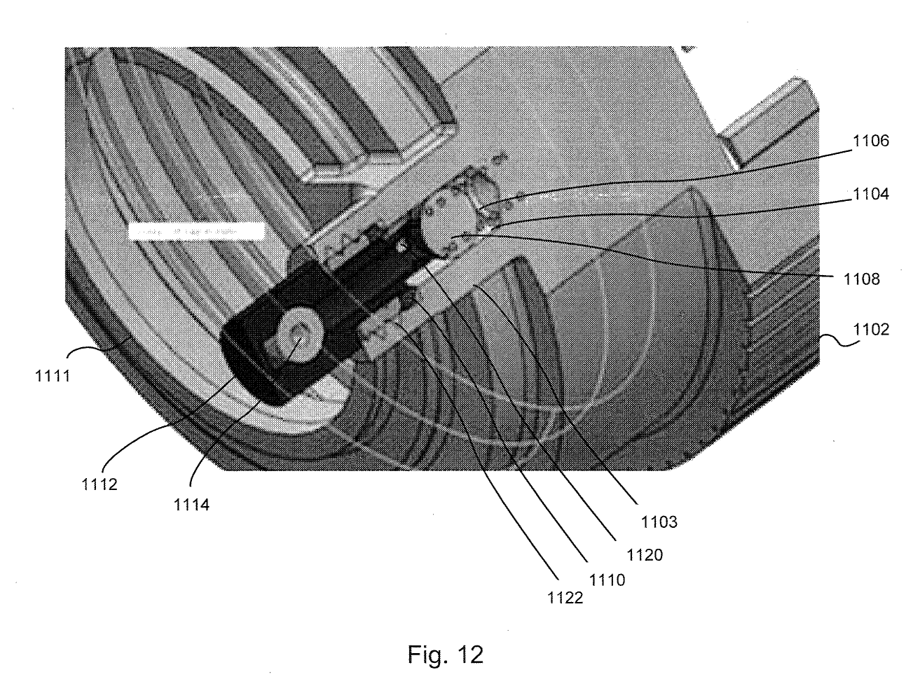

FIG. 12 is a cutaway view of an apparatus providing an additional sanitization fluid dispensing path in accordance with an embodiment of the present disclosure. FIG. 12 may depict sealing housing 1102 of FIG. 11 in which like numbers may correspond to those of FIG. 11. In FIG. 12, adapter 1112 is depicted as inserted into conduit 1104. As illustrated, adapter 1112 has caused sealing stopper 1108 to recede from sealing ring 1110 into conduit 1104 providing access to sanitization fluid via one or more apertures 1120. Adapter 1112 may be secured in conduit 1104 by a locking ring engaging one or more groves 1122. In some embodiments, adapter 1112 may be threaded and may engage a complementary thread inside conduit 1104.

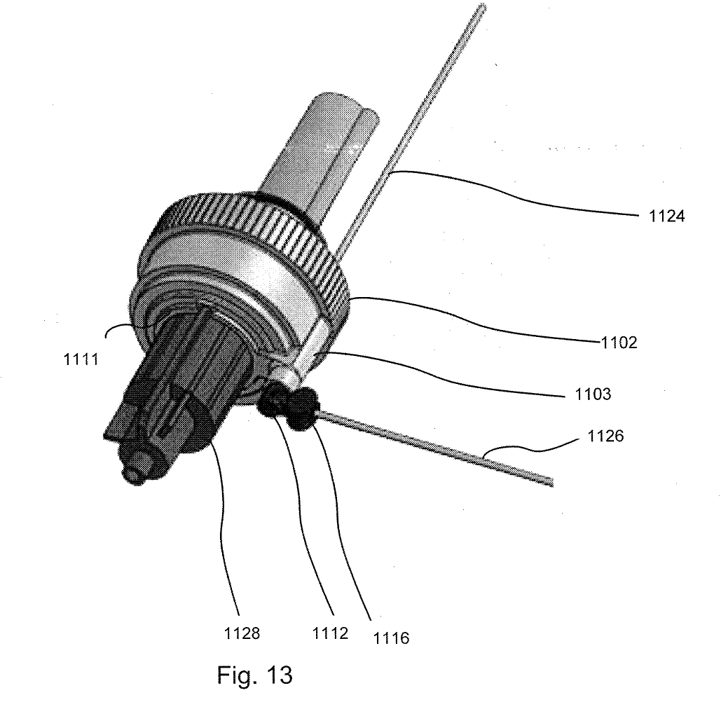

FIG. 13 is an illustration of an apparatus providing an additional sanitization fluid dispensing path in accordance with an embodiment of the present disclosure. FIG. 13 may depict sealing housing 1102 of FIG. 11 without a cut-away view. As depicted in FIG. 13, opening 1111 may accommodate a primary path for sanitization fluid such as, for example, an aspirating nozzle 1128 which may be utilized for a hand sanitization application. Opening 1111 and sealing housing 1102 may be of different configurations, shapes, and diameters to accommodate different manufacturers of sanitization fluid receptacles and/or different uses of a primary path for sanitization fluid via opening 1111. FIG. 13 depicts conduit 1126 attached to mating adapter 1116. Conduit 1126 may be tubing (e.g., medical grade silicon tubing) which may be bonded or otherwise connected to mating adapter 1116. Conduit 1126 may conduct sanitization fluid to one or more separate dispensers (e.g., provide additional sanitization functionality beyond hand sanitization such as sanitization of medical instruments). Conduit 1124 may provide a fluid path from a receptacle to secondary fluid path 1103.

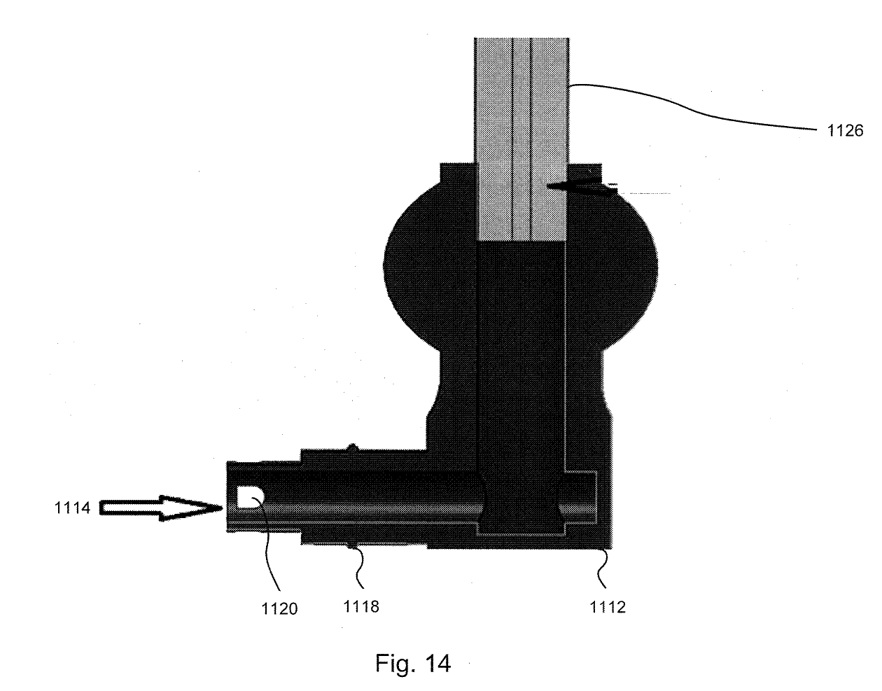

FIG. 14 is a schematic of a sanitization fluid adapter in accordance with an embodiment of the disclosure. Tubing adapter 1112 may, in some embodiments, be manufactured using injected molded plastic. As discussed with respect to FIG. 11, tubing adapter 1112 may include one or more apertures 1120 to conduit 1114. Tubing adapter 1112 may allow conduit 1126 to be inserted partially into conduit 1124 to provide secure attachment and reliable conveyance of fluid. Although depicted with one locking ring 1118 for engaging one or more grooves of a conduit of a secondary fluid path, multiple locking rings may be utilized. In some embodiments, other fasteners may be used (e.g., threads, clips, etc.).

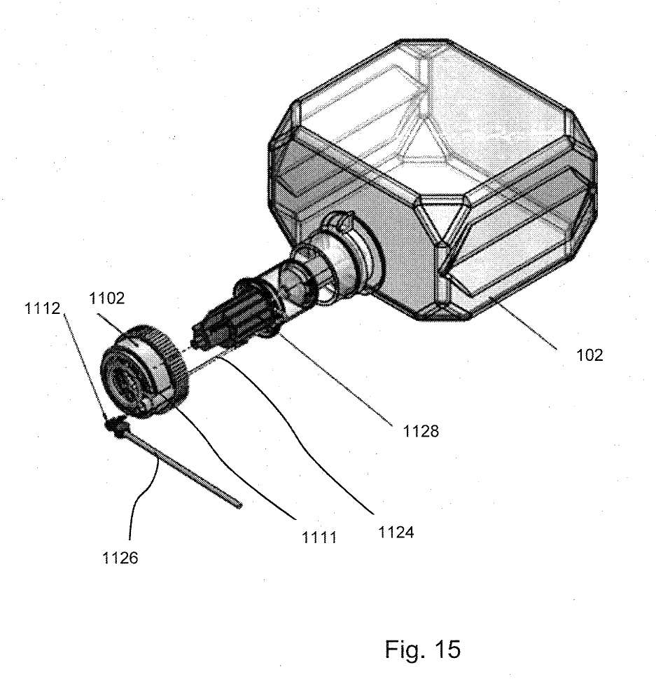

FIG. 15 is an exploded view of an apparatus providing an additional sanitization fluid dispensing path in accordance with an embodiment of the present disclosure. FIG. 15 may depict sealing housing 1102 of FIG. 11 in which like numbers may correspond to those of FIG. 11. FIG. 15 may depict the fit and assembly relationship between an aspirating nozzle 1128 and sealing housing 1102 together with fluid receptacle 102 of FIG. 1. Sealing housing 1102 may contain inner threads or another fastener in opening 1111 on a first side for securing with an outer wall of an opening of receptacle 102. Sealing housing 1102 may contain an inner flange or lip on a second or opposite side for retaining an aspirating nozzle 1128 in opening 1111. Adapter 1112 may fit into conduit 1104 receiving fluid from conduit 1124 and providing fluid to conduit 1126. Although depicted with an aspirating nozzle, other fluid paths may be accommodated. Sealing housing 1102 may be produced in a range of configurations to adapt a secondary fluid path to a variety of receptacles.

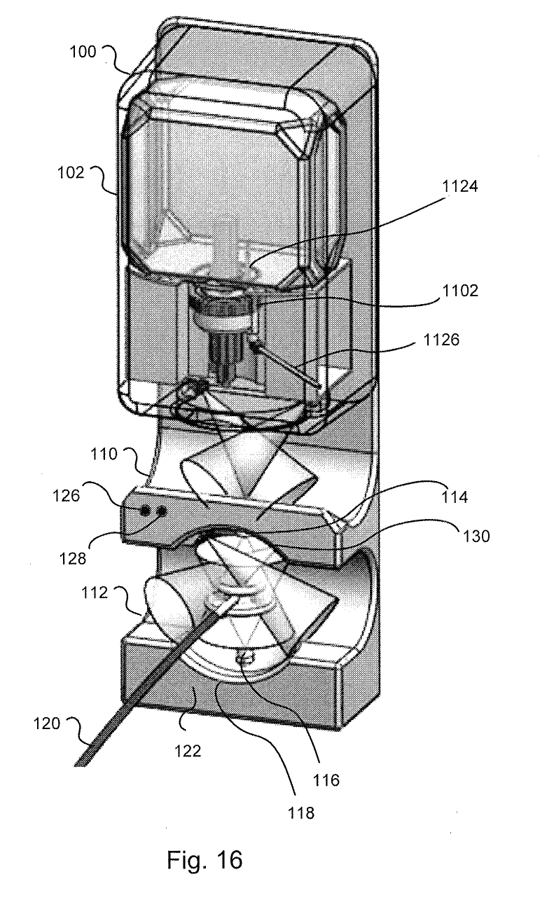

FIG. 16 is an illustration of a sanitization apparatus including an apparatus providing an additional sanitization fluid dispensing path in accordance with an embodiment of the present disclosure. FIG. 16 depicts a sectional view of FIG. 1 to include the sealing housing 1102 installed on receptacle 102. As depicted, conduit 1124 may provide access into receptacle 1102 for a secondary fluid path which may convey fluid to conduit 1126. Although not depicted, conduit 1126 may be connected to other mechanisms (e.g., pump 208 of FIG. 2) for dispensing or application of sanitization fluid.

Although the above description describes embodiments of the invention, it should be understood that the techniques and concepts are applicable to growing systems in general. Thus the invention may be embodied in other specific forms without departing from the spirit or essential characteristics thereof.

While the above describes a particular order of operations performed by a given embodiment of the invention, it should be understood that such order is exemplary, as alternative embodiments may perform the operations in a different order, combine certain operations, overlap certain operations, or the like. References in the specification to a given embodiment indicate that the embodiment described may include a particular feature, structure, or characteristic, but every embodiment may not necessarily include the particular feature, structure, or characteristic.

While the present invention has been described in the context of a system, method or process, the present invention also relates to apparatus for performing the operations herein. This apparatus may be specially constructed for the required purposes, or it may comprise a general-purpose computer selectively activated or reconfigured by a computer program stored in the computer. Such a computer program may be stored in a computer readable storage medium including, without limitation, any type of disk including optical disks, CD-ROMs, and magnetic-optical disks, read-only memory (ROM), random access memory (RAM), magnetic or optical cards, or any type of media suitable for storing electronic instructions.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods, and systems for carrying out the several purposes of the disclosed subject matter. It is important, therefore, that the claims be regarded as including such equivalent constructions insofar as they do not depart from the spirit and scope of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter, which is limited only by the claims which follow.

1. An apparatus providing a sanitization fluid dispensing path comprising:
a sealing housing comprising
a sealing ring, the sealing ring having a first opening for holding a first sanitization fluid dispensing path;
a perimeter ring wall connected at one end to the sealing ring and arranged substantially perpendicular to the sealing ring, wherein the perimeter ring wall is configured to fasten the sealing ring to a sanitization fluid container; and
a sealed cavity configured to accept an adapter to conduct sanitization fluid, wherein acceptance of the adapter opens the sealed cavity to form a second sanitization fluid dispensing path.

2. The apparatus of claim 1, wherein the sealed cavity comprises a channel through the sealing housing having a sealing stopper pressed against an inner rim of a wall of the channel to form a seal, wherein the sealing stopper is configured to recede into the channel upon the acceptance of the adapter into the channel through the inner rim.

3. The apparatus of claim 2, wherein the sealing stopper comprises a ball bearing configured to be held against an O-ring by a retractable retaining mechanism in the channel on an opposite side of the ball bearing from the inner rim.

4. The apparatus of claim 1, wherein the sealed cavity comprises a channel through the sealing housing having a sealing stopper attached to an inner wall of the channel to form a seal, wherein the sealing stopper is configured to break an attachment to the inner wall and to recede into the channel upon the acceptance of the adapter into the channel.

5. The apparatus of claim 4, wherein the sealing stopper comprises a plastic stopper thinner along a perimeter boundary attaching the stopper to the inner wall.

6. The apparatus of claim 1, wherein the sealed cavity comprises a channel through the sealing housing having a sealing membrane capping a first end of the channel and configured to be pierced upon the acceptance of the adapter into the channel.

7. The apparatus of claim 6, wherein the adapter comprises shrouded needle housing.

8. The apparatus of claim 1, wherein the adapter comprises a fluid conduit configured to fit into the sealed cavity and to open the sealed cavity by dislodging a stopper.

9. The apparatus of claim 8, wherein the adapter comprises one or more openings in a side wall of the fluid conduit on a first end of the adapter, wherein the first end of the adapter is configured to fit into the cavity and wherein the adapter further comprises an attachment point at a second end for fluid tubing to conduct sanitization fluid from the fluid conduit.

10. The apparatus of claim 1, wherein the sealed cavity is resealable.

11. The apparatus of claim 1, wherein the sealed cavity is not resealable.

12. A method for providing a sanitization fluid dispensing path comprising:
providing a sealing ring, the sealing ring having a first opening for holding a first sanitization fluid dispensing path;
providing a sealed cavity integrated into the sealing ring wherein the sealed cavity is configured to accept an adapter to conduct sanitization fluid; and
receiving the adapter in the sealed cavity to open the sealed cavity and form a second sanitization fluid dispensing path.

13. The method of claim 12, further comprising providing, via the second sanitization fluid dispensing path, sanitization fluid from a sanitization fluid receptacle to stethoscope sanitation apparatus.

14. The method of claim 12, further comprising providing, via the first sanitization fluid dispensing path, sanitization fluid from a sanitization fluid receptacle to a hand sanitization system.

15. The method of claim 12, wherein providing the sealed cavity comprises providing a channel having a ball bearing configured to be held against an O-ring by a spring in the sealed cavity.

16. The apparatus of claim 15, wherein receiving the adapter comprises receiving a fluid conduit configured to fit into the sealed cavity and to open the sealed cavity by retracting the ball bearing.

17. The method of claim 12, wherein providing the sealed cavity comprises providing a channel through the sealing housing having a sealing membrane capping a first end of the channel and configured to be pierced upon the acceptance of the adapter into the channel.

18. The method of claim 17, wherein receiving the adapter comprises receiving a shrouded needle housing.

19. The method of claim 12, wherein the sealed cavity is resealable.

20. The method of claim 12, wherein the sealed cavity is not resealable.
